(19) United States
(12) Patent Application Publication
(10) Pub. No.: US 2022/0039830 A1
(43) Pub. Date: Feb. 10, 2022

(54) Mooreville Needle Introducer and Corpus Ruler-Dilator

(71) Applicant: Michael Mooreville, Merion Station, PA (US)

(72) Inventor: Michael Mooreville, Merion Station, PA (US)

(21) Appl. No.: 17/393,354

(22) Filed: Aug. 3, 2021

Related U.S. Application Data
(60) Provisional application No. 63/061,102, filed on Aug. 4, 2020.

(51) Int. Cl. A61B 17/34; A61L 29/02; A61B 90/00; A61M 29/00

(52) U.S. Cl. CPC A61B 17/3403; A61B 17/3468; A61B 2017/00367; A61B 90/06; A61M 29/00; A61L 29/02

(57) ABSTRACT

Embodiments for a Mooreville Needle Introducer, and methods for using and making the same are disclosed. The Mooreville Needle Introducer can be constructed of a first part and a second part that can be joined together to form an enclosure for receiving a pusher. The first part and the second part can also be formed with pairs of complementing adapters that fasten together for secure alignment. The first part can be detached from the second part by applying force to a set of handles. When the first part and the second part are detached, they can be effectively cleansed and sterilized, eliminating any lingering bacteria.

### CROSS REFERENCE TO RELATED APPLICATIONS

This application claims priority to U.S. Provisional Patent Application No. 63/061,102, filed Aug. 4, 2020, which is hereby incorporated by reference herein in its entirety.

### FIELD OF INVENTION

The present invention is related to surgical medical devices used with penile implantation procedures, and more particularly, to systems and methods for making and using reusable penile prosthesis implantation tools that are capable of effective sterilization.

### BACKGROUND OF INVENTION

One form of surgical treatment for erectile dysfunction can include the implantation of an inflatable penile prosthesis. The prosthesis implantation procedure generally requires the introduction of an inflatable cylinder within the corpus cavernosus, which is the part of the penis that fills with blood during a normal erection. Two corpora sit side by side within the body of the male genitalia. The implantation procedure generally starts by introducing two separate prosthesis cylinders lying side by side within these corpora.

To introduce these cylinders into the body of the male genitalia, an incision is made in the corpora, approximately halfway in their course through the length of the penis. The prosthesis cylinders, which mimic an erection by filling with fluid, are each pulled within the corpora by a guiding anchor string. Each string is attached to the tip of each prosthesis cylinder.

Currently, these strings are transferred from within the corpora to the outside of the male genitalia by a transferring needle. The transferring needle can be introduced within the corpora using a needle introducer known as the “Furlow needle introducer,” or “Furlow insertion tool.” Once the string is transferred to the outside of the penis through the head of the penis, the prosthesis cylinders can be pulled within the corpora. When pulled completely within the corpora, the prosthesis cylinders fit snugly within the space of the corpora.

The Furlow needle introducer typically includes a small central channel where the transferring needle sits protected until it is ready to be pushed out by a pusher. The pusher is typically constructed to fit within this small channel. The needle introducer also typically includes a small slit extending along its axis to allow the guiding string to lie outside the device. When the needle introducer is removed, the needle introducer can separate from the guiding string and the cylinder through the slit.

Use of the conventional Furlow needle introducer presents several drawbacks. For example, it has been shown that due to the enclosed structural design of the Furlow needle introducer, bacteria can reside within the introducer itself and can be a source of infection, compromising the implanted device. Analyses of cultures from Furlow insertion tools have shown that such tools can represent a source of infection for patients undergoing PP implantation (Yafi, 2020). The bacteria can reside in a number of places of the Furlow insertion tool, including for example, proteinaceous deposits that remain in the central channel, the slit allowing the string to be extroverted, and ridges made on the external surface of the device for measuring the depth of insertion of the device into the corpora.

Further, these micro deposits of bodily fluids or protein are not always removed by standard cleaning procedures, allowing the bacteria to persist and grow worse over time. For example, cleaning by brushing of the exterior surface or passing a cleaning brush through the channel fail to completely remove the micro deposits of body fluids or protein. Over time, any such elements that evade cleansing can harden in locations such as the slit. Once hardened, a shell is formed that can actually shield microorganisms from the sterilizing process that follows cleaning, increasing the risk of infection to the patient. Accordingly, what is needed is a tool that is both reusable and capable of disassembly in a manner that allows for complete and effective cleaning and sterilization such that any residual micro deposits of body fluids or protein are eliminated.

Tools that are used to assist with measurements or dilating tissue during such operations also suffer from several drawbacks. For example, dilators are typically formed to have the same diameter along the length of the dilator body creating a substantially cylindrical shape. Because each dilator has a single diameter, medical professionals that need to operate within a range of diameters typically need a set of dilators to provide the desired range. For example, a medical professional may need diameters that range between 9 and 13 mm. Such a range may require a set of five different dilators to span each millimeter between 9 mm and 13 mm (i.e., 9 mm, 10 mm, 11 mm, 12 mm, and 13 mm). Accordingly, what is needed is a dilator that can both measure the length of the corpus and dilate across range of different diameters.

### SUMMARY OF INVENTION

Embodiments for a Mooreville Needle Introducer, and methods for using and making the same are disclosed. In some embodiments, the Mooreville Needle Introducer can be constructed of a first part and a second part that extend from a distal end to a proximal end and a base at the proximal end comprising a set of handles. The first and second parts can be joined together along their edges to form an enclosure for receiving a pusher. The first part and the second part can be formed with grooves to create a channel that guides a pusher through the enclosure. The first part and the second part can also be formed with pairs of complementing adapters that fasten the first part to the second part and securely align the channel. The first part can be detached from the second part by applying force to the set of handles. The first part and the second part have interior and exterior surfaces that are substantially smooth so that when they are detached, they can be effectively cleansed and sterilized, completely eliminating any lingering bacteria. In some embodiments, the Mooreville Needle Introducer can be constructed of disposable materials and components allowing for single use.

Embodiments for a Corpus Ruler Dilator for measuring and dilating a corpus, and methods for using and making the same are disclosed. The Corpus Ruler Dilator can include a proximal end and a distal end, each having a substantially conical shape for dilating tissue of the corpus. The Corpus Ruler Dilator includes a substantially cylindrical shaft extending from the proximal end to the distal end. The shaft can have a first region at the proximal end and a second region at the distal end, and the diameter of the first region can be larger than that of the second region. The shaft of the Corpus Ruler Dilator can be formed with gradations extending along the axis extending from the proximal end to the distal end while maintaining a continuously smooth surface along the shaft. As the Corpus Ruler Dilator is passed into the patient, the Corpus Ruler Dilator can both measure and simultaneously dilate the tissue of the corpus it is being passed through.

### BRIEF DESCRIPTION OF THE DRAWINGS

The novel features of the invention are set forth in the appended claims. However, for purpose of explanation, several embodiments of the invention are set forth in the following figures.

FIG. 1 is a diagram of a side view of a Mooreville Needle Introducer.

FIG. 2 depicts frontal and perspective views of a clip for a Mooreville Needle Introducer.

FIGS. 3A and 3B illustrates side views of a corpus ruler-dilator.

Like reference symbols in the various drawings indicate like elements.

### DETAILED DESCRIPTION OF THE INVENTION

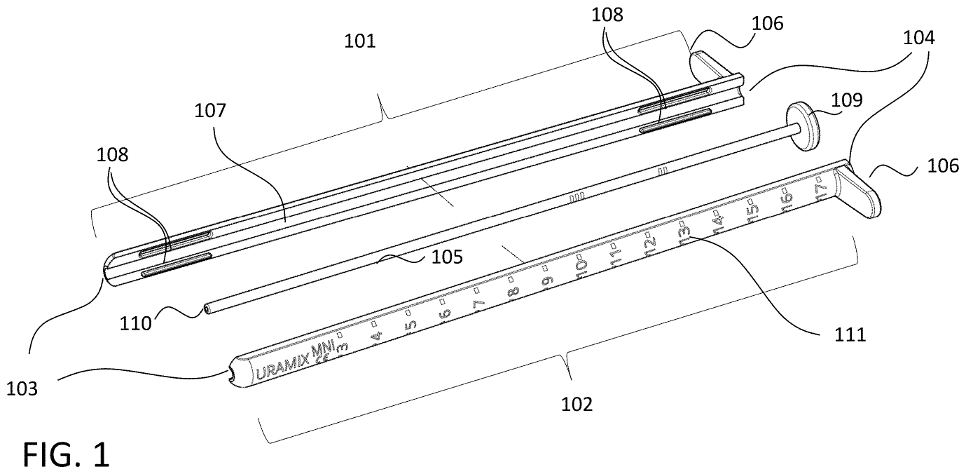

A Mooreville Needle Introducer according to some embodiments of the invention is shown in FIG. 1. The Mooreville Needle Introducer can be formed from a first part 101 and a second part 102. The first part and second part extend from a distal end 103 to a proximal end 104. In some embodiments the shapes of the first and second parts can be mirror image of each other along the axis that extends from the proximal end to the distal end. The first part and second part can be joined along their edges to form an enclosure for receiving a pusher 105, and a base at the proximal end with at least one or more handles 106.

The interiors of the first part and second part can be formed with grooves 107. When the first and second parts are joined along their edges, the grooves along the interior create a channel. The channel can be used to guide the pusher through the enclosure.

The exterior of the second part can be formed with gradations 111 for taking measurements during the procedure. For example, the gradations can indicate depth along the corpora. The gradations can be engraved in a manner that maintains a continuously smooth surface along the exterior and interior of the shaft of the Mooreville Needle Introducer. Thus, the markings leave no indentations or ridges on the interior or exterior surface. By maintaining a smooth surface on the exterior of the tool, the risk of micro deposits of proteinaceous materials, bodily fluids, or proteins forming is greatly reduced and lingering bacteria can be completely eliminated.

The first part and second part can be detached and reattached along the axis that stretches from the proximal end to the distal end. The two handles allow for easy separation and detachment of the first and second parts and subsequent cleaning of the introducer.

In some embodiments, the first part and second part can be formed to detach and reattach along other axes. For example, the first part and second part can be formed to detach and reattach along an axis orthogonal to the axis stretching from the proximal end to the distal end.

The first and second parts include complementing male and female adapters 108. The male and female adapters can be formed as pairs on the proximal and distal ends of the introducer on opposite sides of the channel. When the first and second parts are joined together, the male adapter joins with the female adapter ensuring that the two parts are secured and the channel stays aligned on the axis extending from the distal end to the proximal end.

In some embodiments of the invention, the male and female adapters can be formed to include hooks, pins, or snap fasteners to enhance the fastening of the first and second parts and ensure the first and second parts are secured and that the channel stays aligned on its axis. When joined, the female adapter and the male adapter are aligned so that the groove formed by the first part is fully aligned with the groove formed in the second part, creating a seamlessly formed channel to receive a pusher.

The complementing adapters can be detached by applying force or pressure to the handles at the base of the introducer. In this way, the Mooreville Needle Introducer allows for easy detachment and disassembly. In some embodiments, a push-button and release latch can also be used to ensure alignment and easy release when the device needs to be disassembled for sterilization.

As described above, the first and second parts can be formed with respective grooves that form a channel for receiving a pusher. The pusher can include a flange end piece 109 at its proximal end and a tip 110 at the distal end for pushing a needle through the channel.

In some embodiments, the first and second parts can be 18 cm in length from the distal end to the proximal end. The pusher can have substantially similar length as the enclosure formed by the first and second parts.

In some embodiments, the first and second parts can be formed with entirely smooth surfaces, avoiding the creation of any ridges or crevices that may harbor proteinaceous material. The pusher can be formed with a smooth surface, void of any indentations or ridges by marking the gradations through laser etching processes on the surface. In this way, the first and second parts are void of any hidden internal or external surfaces, which could harbor proteinaceous material.

In some embodiments, the first and second parts, and the pusher are all constructed from surgical steel. The steel first and second parts and pusher that form the Mooreville Needle Introducer can be washed and then sterilized by, for example, autoclave. After washed and sterilized, the Mooreville Needle Introducer can be reused with minimal risk of bacteria lingering in any deposit or crevice of the tool. In other embodiments, the first and second parts and pusher can be formed of a medical grade hard plastic material and the Mooreville Needle Introducer can be intended for single use.

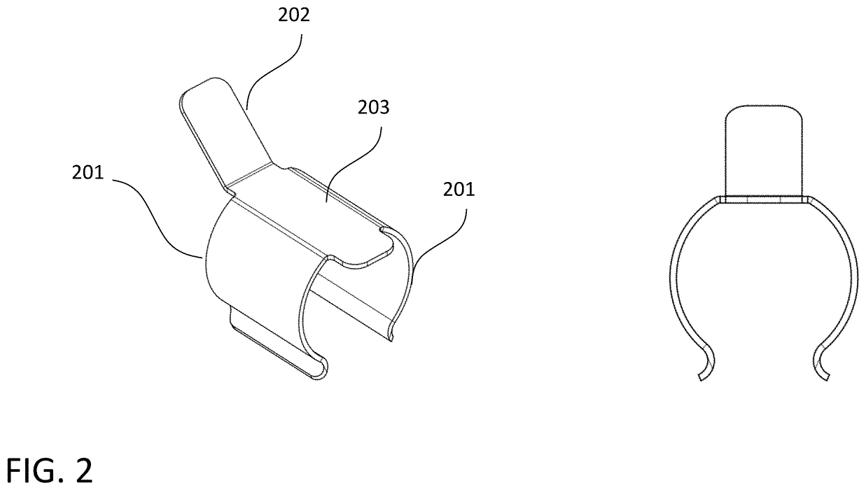

A clip for a Mooreville Needle Introducer according to some embodiments of the invention is shown in FIG. 2. The clip can include opposing arms 201 that substantially encase the circumference of the Mooreville Needle Introducer and form an opening for enclosing the first and second parts of the Mooreville Needle Introducer. When the Mooreville Needle Introducer is inserted through the opening formed by the opposing arms, the first and second parts of the Mooreville Needle Introducer are firmly attached to each other and cannot be separated until the clip is removed. In this way, the clip provides improves the strength of the grip between the first and second parts and reduces the risk of any bacteria or deposit finding an opening to seep through. The clip can include a substantially flat section 203 in between the opposing arms from which a tab 202 extends. The tab can extend from the flat section at angle so that a user can push the tab to glide it over the first and second parts of the Mooreville Needle Introducer.

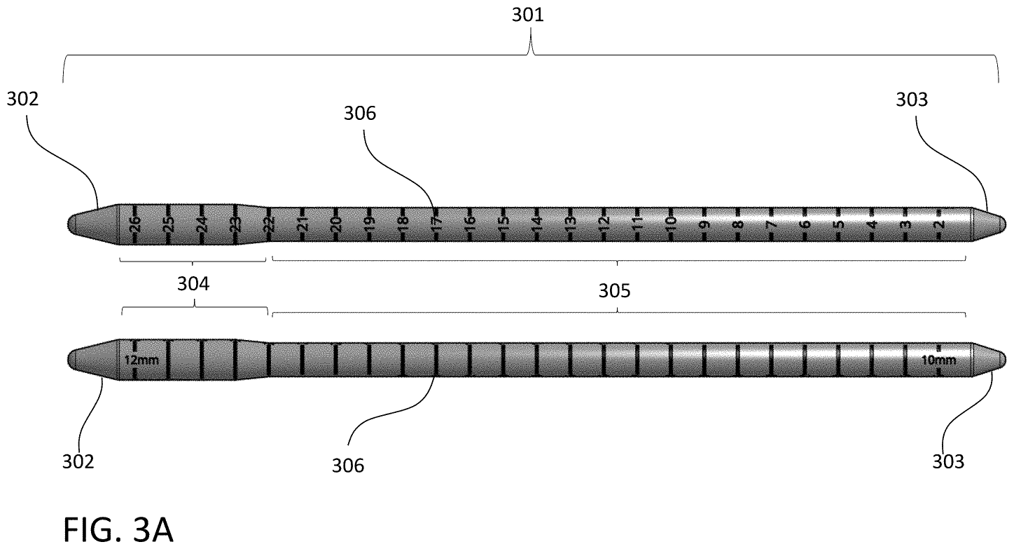

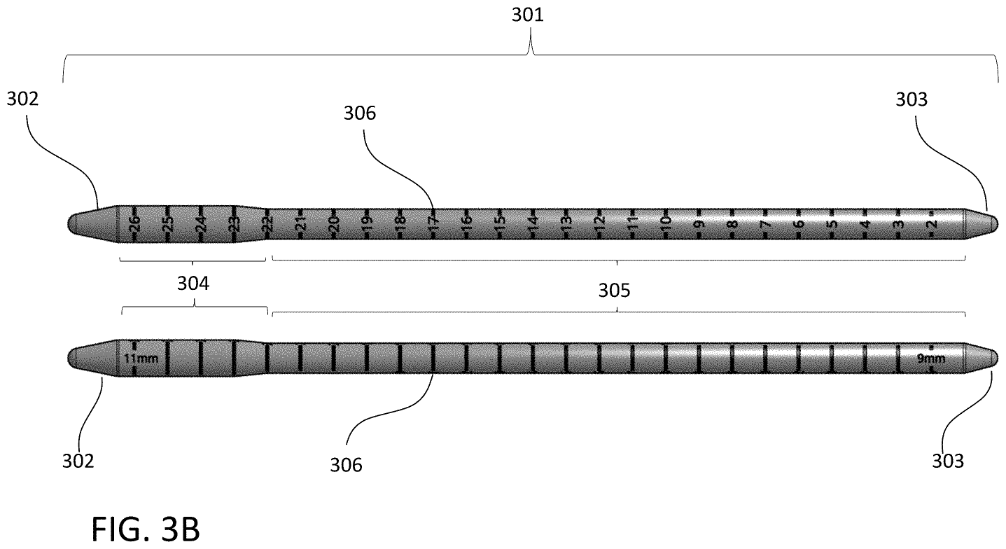

Corpus Ruler-Dilators according to some embodiments of the invention are shown in FIGS. 3A and 3B. A Corpus Ruler-Dilator can be used to measure and dilate the corpora to determine the size of the prosthesis cylinders that fits most comfortably and appropriately within the patient's genitalia.

The Corpus Ruler Dilator 301 can include a proximal end 302 and a distal end 303. The proximal end can be used for inserting into a corpus and is located opposite the distal end. The Corpus Ruler Dilator can further include conical tips at the proximal and distal end that dilate and stretch the corpora as they pass into, or out of the genitalia of the patient.

The Corpus Ruler Dilator includes a shaft that extends from the proximal end to the distal end. In some embodiments the shaft of the Corpus Ruler Dilator can be substantially cylindrical along the axis that extends from the distal end to the proximal end. In some embodiments the diameter of the shaft can vary along this axis. For example, the shaft can have a first region 304 of the Corpus Ruler Dilator at the proximal end, a second region 305 extending towards the distal end of the Corpus Ruler-Dilator, and the diameter of the first region can be larger than the diameter of the second region. Such an exemplary Corpus Ruler Dilator is shown in FIG. 3A, where the first region has a diameter of 12 mm and a second region with a diameter of 10 mm. The exemplary Corpus Ruler Dilator shown in FIG. 3B has a first region with a diameter of 11 mm and a second region with a diameter of 9 mm.

In some embodiments (not pictured), the Corpus Ruler-Dilator can have additional regions along the shaft with varying diameters. For example, the Corpus Ruler-Dilator can have a third, fourth, and fifth region and the diameter of each region can increase from the distal end of the shaft to the proximal end. Each region can, for example, correspond to a diameter in the range between 9 and 13 mm. For example, the Corpus Ruler-Dilator can have a diameter of 9 mm along its smallest region and a diameter of 13 mm in its greatest diameter. In some embodiments the region with the smallest diameter has a conical tip that can be used as the dilating head to dilate tissue as it is being passed into the patient. The region with the largest diameter can similarly have a conical tip for dilating tissue that is being passed out of the patient.

The lengths of the first and second regions along the axis extending from the distal end to the proximal end can be equal or unequal. For example, the length of the first region along the axis can be shorter than the length of the second region along the same axis, as shown in FIGS. 3A and 3B. In other embodiments (not pictured), the length of the first region can be greater than the second region, or the two regions can have equal length. Where the shaft includes additional regions, the regions can likewise have lengths that are equal, or unequal.

In some embodiments of the invention, the Corpus Ruler-Dilator can include gradations 306 formed on the shaft extending along the axis extending from the proximal end to the distal end. The gradations can be used to measure the length of the corpora as it is being inserted. In this way, the Corpus Ruler-Dilator can simultaneously dilate and measure the corpus as it is being inserted into the patient.

In some embodiments, the Corpus Ruler-Dilator can include gradations on opposite sides of its shaft. The gradations can be engraved in a manner that allows for a continuously smooth surface along the shaft of the Corpus Ruler-Dilator. The markings can be engraved in a manner that leaves no surface indentations. As described above, maintaining a smooth surface on the exterior of the tool significantly reduces the risk of micro deposits of proteinaceous materials, bodily fluids, or proteins from forming.

The proximal and distal ends of the Corpus Ruler-Dilator can have dilating head that are conical to allow for its insertion as well as dilation to 9 mm. The length of the corpus can be determined by referencing the gradations on the body of the Corpus Ruler-Dilator. The proximal end can then be passed through the corpus to dilate the tissue to 11 mm diameter. Dilation to 11 mm is sufficient to allow the prosthesis cylinder to be inserted within the corpus cavernosus.

In some embodiments, the Corpus Ruler-Dilator includes both the varying diameters as well as the gradation markings. In this way, the Corpus Ruler-Dilator can combine functions of six different tools (one measuring and five dilators of differing diameters) into one tool.

In some embodiments, the Corpus Ruler-Dilator can be constructed of surgical steel. Like embodiments of the Mooreville Needle Introducer, the steel Corpus Ruler-Dilator can be effectively cleaned and sterilized for reuse.

### REFERENCES

“Prospective analysis of culture from the Furlow insertion tool: a possible etiology for penile prosthesis infections”, (Faysal A. Yafi, et al, International Journal of Impotence Research, 2020, https://doi.org/10.1038/s41443-020-0256-2).

What is claimed is:

1. A needle introducer comprising:
a first part that extends from a distal end to a proximal end;
a second part that extends from the distal end to the proximal end;
a base at the proximal end comprising a set of handles,
wherein the first part and the second part are configured to be joined along their edges to form an enclosure for receiving a pusher,
the first part and the second part are formed with grooves to create a channel that guides a pusher through the enclosure,
the first part and the second part are formed with pairs of complementing adapters that fasten the first part to the second part and securely align the channel,
the first part and the second part are detachable by applying force to the set of handles, and
the first part and the second part have interior and exterior surfaces that are substantially smooth to enable effective cleaning and sterilization of the needle introducer.

2. The needle introducer of claim 1 wherein the needle introducer is constructed of surgical steel to allow for reuse

3. The needle introducer of claim 1 wherein the needle introducer is constructed of a medical grade plastic.

4. The needle introducer of claim 1 wherein the set of handles further comprises a push button and release latch.

5. The needle introducer of claim 1 wherein the complementing adapters comprise hooks.

6. The needle introducer of claim 1 further comprising gradations wherein the gradations are engraved to maintain a continuously smooth surface along the needle introducer.

7. The needle introducer of claim 1 wherein the surface of the pusher is formed with a continuously smooth surface for effective cleaning and sterilization.

8. The needle introducer of claim 7 wherein the pusher is constructed of surgical steel.

9. The needle introducer of claim 1 further comprising a clip.

10. A corpus ruler dilator for measuring and dilating a corpus, the corpus ruler dilater comprising:
a proximal end having a substantially conical shape for dilating tissue of the corpus;
a distal end having a substantially conical shape for dilating tissue of the corpus; and
a shaft extending from the proximal end to the distal end, the shaft being substantially cylindrical and having a first region at the proximal end and a second region at the distal end, wherein the diameter of the first region is larger than the second region;
wherein the shaft is formed with gradations extending along the axis extending from the proximal end to the distal end, and
wherein the gradations are engraved to maintain a continuously smooth surface along the shaft.

11. The corpus ruler dilator of claim 10, wherein:
the first region has a diameter of 12 mm, and
the second region has a diameter of 10 mm.

12. The corpus ruler dilator of claim 10, wherein:
the first region has a diameter of 11 mm, and
the second region has a diameter of 9 mm.

13. The corpus ruler dilator of claim 10, wherein the shaft further comprises a third, fourth, and fifth region, wherein the diameter of each region increases from the distal end of the shaft to the proximal end.

14. The corpus ruler dilator of claim 13, wherein:
the first region has a diameter of 13 mm,
the second region has a diameter of 12 mm,
the third region has a diameter of 11 mm,
the fourth region has a diameter of 10 mm, and
the fifth region has a diameter of 9 mm.

15. The corpus ruler dilator of claim 10, wherein the length of the first region along the axis extending from the distal end to the proximal is shorter than the length of the second region along the same axis.

16. The corpus ruler dilator of claim 14, wherein the lengths of the first, second, third, fourth, and fifth regions along the axis extending from the distal end to the proximal increase from the first region to the fifth region.

17. The corpus ruler dilator of claim 10, wherein the corpus ruler dilator is a reusable corpus ruler dilater constructed from surgical steel.
